(19) United States
(12) Patent Application Publication
(10) Pub. No.: US 2004/0029200 A1
(43) Pub. Date: Feb. 12, 2004

(54) Method and kits for the detection of renal cell carcinoma in a biological fluid of a patient

(76) Inventor: Thomas Weimbs, Shaker Heights, OH (US)

Correspondence Address: Pepper Hamilton LLP, One Mellon Center, 50th Floor, 500 Grant Street, Pittsburgh, PA, 15219, US

(21) Appl. No.: 10/444,639

(22) Filed: May 22, 2003

Related U.S. Application Data
(60) Provisional application No. 60/382,681, filed on May 22, 2002.

(51) Int. Cl. G01N 33/574

(52) U.S. Cl. 435/7.23

(57) ABSTRACT

The present invention relates to novel methods for detecting and evaluating urological cancers and in particular renal cell carcinoma (RCC). The methods of the present invention are based on the discovery that extracellular matrix proteins are absent or decreased in the urine of patients-with RCC and are diagnostic markers for the detection of RCC. Furthermore, the methods of the present invention are based on the discovery that metalloproteinases (MP) activity determined by extacellular matrix protein degradation is increased in patients with urological cancers and are diagnostic markers for the detection of these cancers. Diagnostic agents and methods for detecting the presence of RCC in biological samples such as urine are disclosed. A kit for detecting the extracellular matrix proteins, degradation fragments of extracellular matrix proteins, and activity of MPs are also disclosed.

### CROSS REFERENCES TO RELATED APPLICATIONS

This application claims priority from U.S. Provisional Application Serial No. 60/382,681 filed May 22, 2002 the contents of which are incorporated herein by reference in their entirety.

### BACKGROUND OF THE INVENTION

For several forms of solid tumors, the process of tumor detection and staging has been greatly improved by the development of assays that detect and measure tumor-specific markers in specimens of patient tissues or body fluids. An example of this type of technology is exemplified by the use of prostate specific antigen (PSA) screening for prostate cancer. Such assays have the potential of revolutionizing the clinical approach to diagnosis, staging and monitoring the effect of therapeutic intervention in human malignancies. Many of these tumor marker assays are based on immunologic detection of the tumor marker protein.

There are a number of cancers which are difficult to detect in the early stages of the disease. Renal cell carcinoma remains a disease that would benefit tremendously from improvements in early detection. Renal cell carcinoma is the third most common malignancy of the urinary tract after prostate and bladder cancer. This condition is usually asymptomatic until a relatively advanced state is reached. Consequently a large proportion of new patients already have metastatic disease at initial diagnosis. In recent years the incidental discovery of asymptomatic renal cell carcinoma has significantly increased due to more widespread use of imaging techniques. A much higher percent of incidentally discovered renal cell carcinomas are localized and of low grade compared with symptomatic tumors. Renal cell carcinoma is usually completely curable by radical or partial nephrectomy provided that the tumor is localized. Mainly due to the increase in incidental discovery the 5-year survival rate has increased to above fifty percent (50%) from much lower outcomes in the 1970s and 1980s.

At present, no clinically relevant markers for the early detection of renal cell carcinoma (RCC) are available. The ability to diagnose early, asymptomatic renal tumors would have a very high impact on improving the outcome of RCC. The availability of an effective diagnostic assay may make it possible to screen routinely high and/or low risk populations. The former includes patients with end stage renal disease and acquired renal cystic disease in whom the risk of renal cell carcinoma is up to 100-fold higher than in the general population. Furthermore, hereditary forms involving renal cell carcinoma, such as von Hippel-Lindau disease and hereditary papillary renal cell carcinoma, are in this category. A sensitive renal cell carcinoma screening test would also have high value for detecting tumor recurrence in patients with renal cell carcinoma after total or partial nephrectomy. An ideal screening assay would be inexpensive to screen asymptomatic populations, noninvasive, and could be developed into a high throughput assay relying on commonly available clinical diagnostic equipment and expertise.

### SUMMARY OF THE INVENTION

In one embodiment of the present invention, the methods, devices, and/or kits of this invention involve detecting the absence or decrease of extracellular matrix proteins in biological fluids, particularly in urine in order to facilitate the diagnosis of the subject for cancer, particularly urological cancers, and most particularly renal cell carcinoma.

In another embodiment of the present invention, the methods involve detecting the presence of proteolytic activity for the degradation of extracellular matrix proteins in order to facilitate the diagnosis of the subject for carcinoma.

In an alternative embodiment of the present invention, metalloproteinases or their byproduct are detected to facilitate the diagnosis of the subject for cancer, particularly urological cancers, and most particularly renal cell carcinoma.

Detection of extracellular matrix proteins, proteases, or proteolytic activity in urine may be by electrophoresis, immunological assays, or assays that detect the degradation of substrates. Examples of these methods are western blot, enzyme-linked immunosorbant assay, in-gel zymography, or assays based on fluorescence dequenching of labeled substrates.

Embodiments of the present invention also features kits for facilitating diagnosis and prognosis of cancer, the kits including a container; a reagent for detecting proteolytic activity, an enzyme, or a protein in a urine sample; and instructions. In a preferred embodiment of the kit, renal cell carcinoma is being detected.

Although the various embodiments of the preent invention may be used as a sensitivite means to detect, monitor or diagnose both urological and non-urological (e.g. via glomerular filtration of plasma) cancers, the embodiments described herein are particularly suitable for urological cancers (e.g. bladder, renal, and prostate). Certain embodiments of the present invention are particularly suitable for renal cell carcinoma.

### BRIEF DESCRIPTION OF THE FIGURES

) FIG. 1 illustrates an analysis of urinary ECM proteins: A, concentrated urine samples corresponding to equal original volumes from patients with renal cell carcinoma or controls were separated by SDS-PAGE, followed by immunoblotting with antibodies against collagen IV (Col-IV), fibronectin (FN) or laminin (LN); Regions of full-length proteins are shown. Values above lanes indicate patient and control numbers; +, positive controls (human serum for fibronectin, and total human kidney lysate for laminin and collagen IV); FIG. 1B, excretion profile of collagen IV in consecutive urine specimens collected at different times from healthy volunteer.

FIG. 2 illustrates total urinary protein patterns: Concentrated urine samples corresponding to equal original volumes from patients with renal cell carcinoma (RCC) or controls were separated by SDS-PAGE, followed by staining with Coomassie blue. Values indicate molecular weight markers in kDa.

FIG. 3 illustrates proteolytic activity for in vitro degradation of fibronectin in renal cell carcinoma (RCC) urine: Purified human fibronectin was incubated with concentrated renal cell carcinoma or control urine specimens from 3 patients and 2 controls for indicated times; Disappearance of full-length fibronectin was monitored by Western blot analysis.

FIG. 4 illustrates detection of MP activity using fluorescence based microtiter plate assay: A, concentrated urine samples from patients and controls were mixed in 96-well plates in duplicate with reaction buffer containing fluorescent substrate, namely collagen IV modified with fluorescein; Proteolytic activity was indicated by increased fluorescence, which was monitored with fluorescence microplate reader. Background was subtracted and average fluorescence values were plotted for patients and controls; Values of duplicate measurements varied by less than 10%. Investigated specimens were from groups of patients/controls different from those shown in Table 1. Average value for renal cell carcinoma was 3.4-fold higher than for controls (t test p<0.0001). Difference was also significant by Wilcoxon and median tests. RCC, renal cell carcinoma. B, standard curve measured using collagenase IV from Clostridium histolyticum under otherwise identical assay conditions shows fluorescence values within linear range of assay. C, in 5 randomly selected renal cell carcinoma urine specimens zinc dependency was investigated by including 2.5 mM. 1,10-phenanthroline, which resulted in almost complete inhibition of proteolytic activity.

FIG. 5 illustrates Gelatinolytic activity in renal cell carcinoma and control urine specimens was analyzed by in-gel gelatin zymography: Molecular weight assignment was only approximate because reduced marker proteins were expected to show different migration behavior than nonreduced proteins in urine. White bands indicate gelatinases. Values indicate molecular weight in kDa;

FIG. 6 illustrates fluorescence activity assay results for subjects having prostate cancer, bladder cancer, Stone disease, and healthy controls from concentrated urine specimens from groups of patients/controls different from those shown in Table 1 and FIG. 4A

### DETAILED DESCRIPTION OF THE INVENTION

It appears that urine from patients with cancer, even at early stages contains metalloproteinase (MP) activity, which leads to the degradation of normally excreted extracellular matrix (ECM) proteins. As is discussed in greater detail herein, the presence of MP activity and absence of ECM proteins in urine can be exploited to develop a practicable screening assay for renal cell carcinoma detection. Examples of extracellular matrix proteins include, but are not limited to, collagen IV, fibronectin, and laminin.

A preferred method for detecting asymptomatic tumors should include the following characteristics. First, the procedure should be non-invasive. This eliminates the need for biopsy, blood test, etc. Second, it should be possible to perform the procedure in any hospital or clinical lab using standard equipment and expertise. This eliminates specialized methods such as PET scanning, microsatellite analysis, etc. The procedure is preferably inexpensive, which eliminates the other procedures such as ultrasound, CT, and MRI. Finally, the specificity of the procedure should be very high (preferably greater than 85%, even more preferably greater than 90% and even more preferably closer to 100%. In this regard, the present invention provides a suitable procedure by providing a microtiter plate assay to detect presence/absence of a protein in urine.

Investigation of extracellular matrix (ECM) proteins in urine of RCC patients and healthy controls revealed that these proteins are readily detectable in control urine but significantly decreased or absent in patient urine as summarized in Table 1. An increase in active forms of metalloproteinase in the urine correlates to the presence of renal cell carcinoma in a subject; the absence of extracellular matrix proteins in urine correlates with the presence of renal cell carcinoma in the subject. The sensitivity and specificity of an assay designed to detect extracellular matrix proteins to indicate the presence of, for example, RCC is very high. Positive diagnosis of RCC was determined if at least two ECM's are undetectable. If at least two ECM protein are absent from the urine of the patient, the sensitivity of the diagnosis of RCC for a mixture of males and females is 95%. The specificity of the diagnosis of RCC for a mixture of males and females is 95%. In a preferred embodiment of the present invention, an absence or decrease of extracellular matrix proteins in the urine of patients is used as a marker of RCC.

In a further embodiment of the present invention, the presence of degradation products of extracellular matrix proteins in the urine of patients is used as a marker of certain cancers, both urological and non-uroligical.

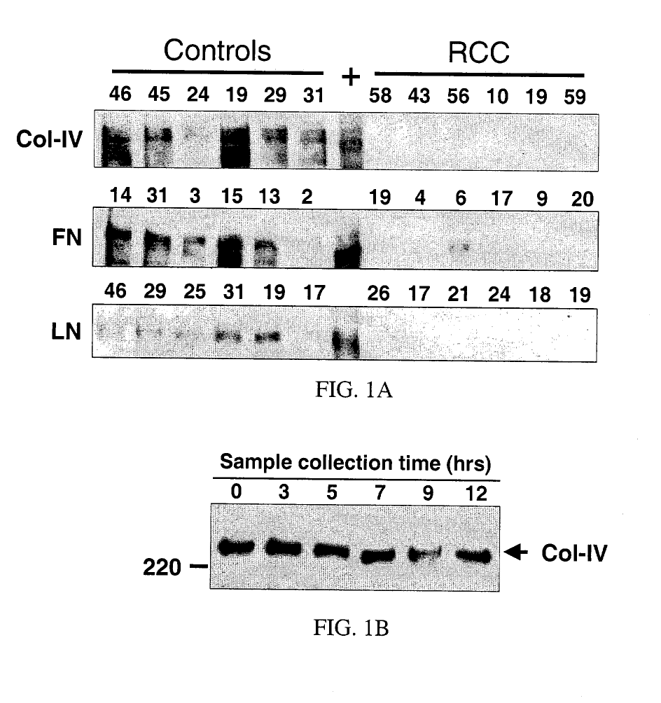

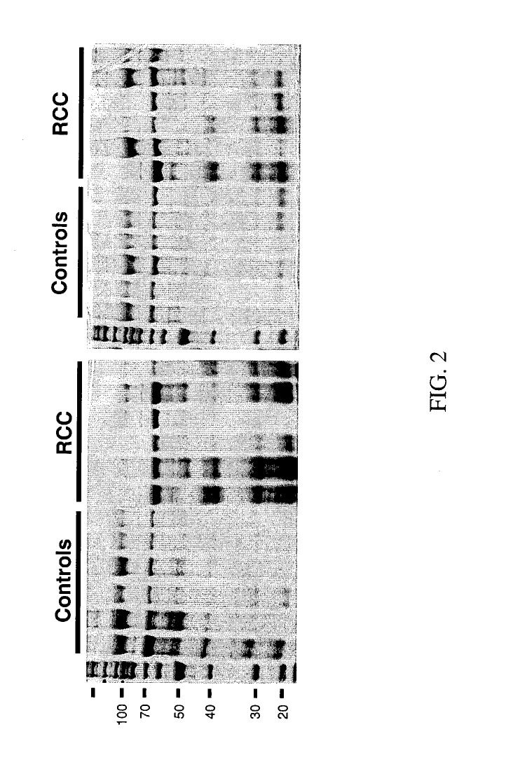

The overall patterns of excreted proteins do not differ significantly between patient and control urines, FIG. 2, indicating the ECM proteins are specifically affected in cancer patients, particularly RCC patients. All urine specimens were collected at random times and processed immediately. The possibility that the excretion of ECM proteins may vary depending on the time of specimen collection was excluded. The absence of ECM proteins appears to be due to increase excretion of metalloproteinases (MPs) in the urine of the patients.

Metalloproteinases (MPs) are a family of closely related metal-dependent endopeptidases. For example, gelatinase A (MP-2 or 72 kDa gelatinase/type IV collagenase) and gelatinase B (MP-9 or 92 kDa gelatinase/type IV collagenase) have been identified as playing a major role in cancer invasion and metastasis.

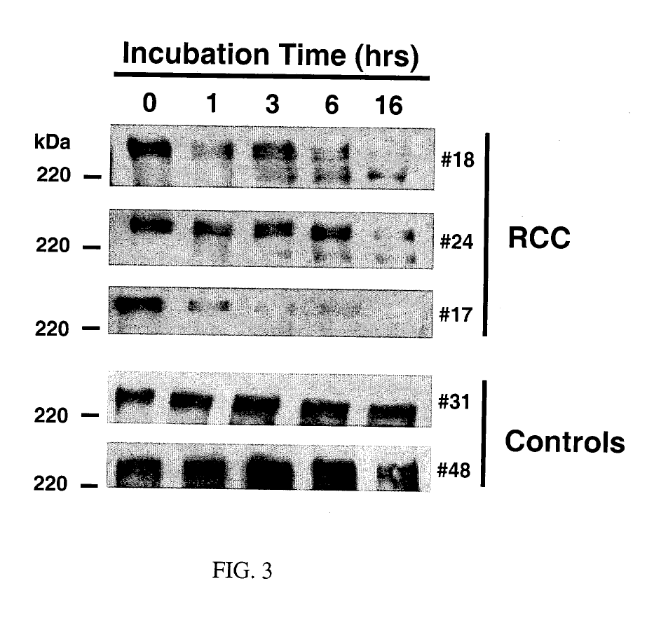

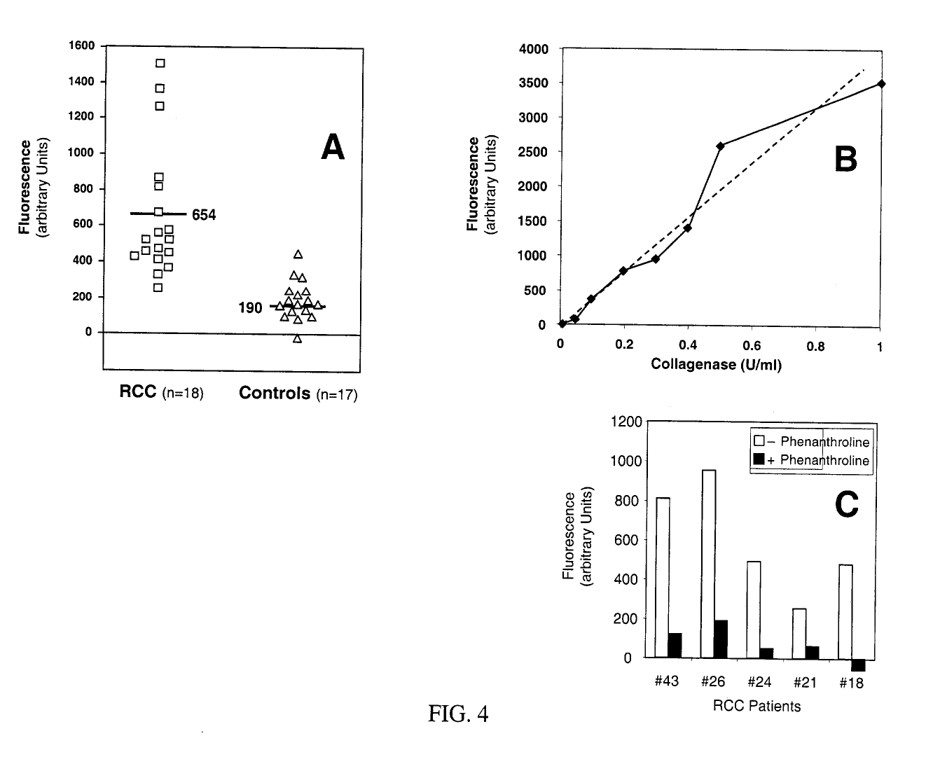

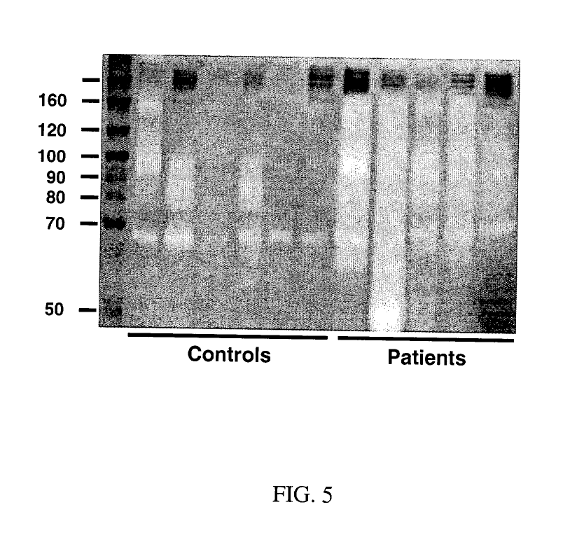

Incubation of purified ECM proteins with urine from RCC patients leads to their degradation as shown as an example in FIG. 3. This indicates the presence of proteolytic activity specific for ECM proteins in the urine of subjects with RCC. The presence of degradation products of the extracellular matrix protein after incubation with the urine of a subject correlates with an increase in the active form of metalloproteinases in the subject and an indication of renal cell carcinoma in the subject. This can be further demonstrated by other commonly used methods to detect proteolytic activity, especially the activity of MPs. As an example, gelatin zymography, FIG. 5, shows increased gelatinase activity in RCC urines. Furthermore, fluorescent-labeled substrates such as collagen-IV can be used in a microtiter-plate assay to detect MP activity due to fluorescence-dequenching as a result of proteolytic cleavage as illustrated in FIG. 4.

In a preferred embodiment of the present invention, highly specific and reliable diagnostic agents and methods are provided to determine the presence of renal cell carcinoma. One embodiment of the present invention is the detection of MP or its activity excreted in the urine of the patient as a marker of the disease. In a preferred embodiment, the detection of MP activity is a fluorescense-based microtiter plate assay.

The present invention includes diagnostic agents and methods useful in the detection of cancers using enzymes, such as MPs, or proteins, such as ECM, excreted in the urine as markers of the disease. The observations provided herein can be used for many different strategies to detect the presence of cancers using urine samples, including: monitoring the absence or degree of decrease of undegraded ECM proteins; monitoring the presence of MPs; and monitoring the activity of MPs. Importantly, in preferred embodiments of the present invention, it is determined whether the MPs present in the urine are active or not (e.g complexed).

To detect the absence or decrease of undegraded ECM proteins in urine several methods are available. One example is the detection of ECM proteins by western blot analysis using specific antibodies. Western blot is a very commonly used method, and several widely known variations are known to those skilled in the art. Many antibodies (polyclonal or monoclonal) against a variety of ECM proteins are commercially available or can be raised using standard procedures by those skilled in the art. By comparing the amount the extracellular matrix proteins in the urine of the patient to the mean amount the extracellular matrix proteins in a control group or the normal population, a decrease in the amount of the extracellular matrix protein in the subject's urine may be used as an indication of increased probability of a urological cancer and in particular a renal carcinoma. Another example of the detection of undegraded ECM proteins makes use of antibodies that detect the sites of proteolytic cleavage within the given ECM protein. Such antibodies can be raised against synthetic peptides and can be used using commonly known immunological methods such as western blot or ELISA.

To detect the presence of degradation products of ECM proteins in urine, several methods are available. One example is the use of specific antibodies against a given ECM protein fragment in western blot analyses. Another is the use of antibodies raised against peptides of the newly created N- or C-termini of the proteolytic sites (so-called neo-antigens) which can be used in techniques such as western blot or ELISA.

To detect the activity of MPs in urine, several methods are available. One example is the monitoring of fluorescence dequenching that is the result of proteolytic degradation of a fluorescently labeled substrate (such as collagen-IV) that is mixed with the urine sample. This method, and variations of it, is well known to those skilled in the art. Variations can include the use of different substrates, or different fluorophores. The amount of active metalloproteinases detected by this or other methods may be compared to the mean amount of active metalloproteinases in a control group of healthy patients or to the normal population. An increase in the amount of active metalloproteinases in the subject's urine being an indication of an increased probability of a renal cell carcinoma as illustrated in FIG. 4A. To detect the presence of MPs in urine, several methods are available. One example is the use of specific antibodies against a given MP in western blot analyses or ELISA.

MP will be used as a representative enzyme or protein in the following description of the invention. The invention includes a first substance capable of immunologically reacting with enzymes broadly described as metalloproteinases (MPs). Such MPs include, but are not limited to, gelatinase A (72 kDa type IV collagenase/gelatinase (MP-2), gelatinase B (92 kDa type IV collagenase/gelatinase (MP-9)), stromelysin (MP-3), partial breakdown products of these proteins and combinations thereof.

The first immunologically-reactive substance is preferably a monoclonal antibody having specificity for one or more MP enzymes or breakdown products of MPs. Examples of such antibodies include monoclonal murine anti-MP-2, monoclonal murine anti-MP-9 and monoclonal murine anti-MP-3, as set forth by Bergmann, et al. J. Clin. Chem. Clin. Biochem 27, 351-359 (1989) or Cell Tech Lmt, (Slough, England). Alternatively, antibodies such as rabbit polyclonal antibodies to native MPs or peptide components such as peptide sequences of the native MPs may be used.

The present invention also includes a method of detecting renal cell carcinoma in a urine sample and a diagnostic kit. The method includes contacting a diagnostic agent such as that set forth above, with the biological sample and measuring the total amount of MP present to determine whether renal cell carcinoma is present. Preferably, the method or diagnostic kit is able to distinguish between the level of endogenous activity of the MPs, rather than simply detecting the presence or absence of the MPs. A kit for detecting cancer in a subject may include instructions; a reagent including a buffer and a fluorescent substrate; well plates; and a sterile container for combining the reagent with urine from the subject to form a mixture. The mixture of the fluorescent substrate buffer and urine is deposited into the well plates. The presence of fluorescence of the mixture in the well plates is used as an indication that the subject has urological cancer.

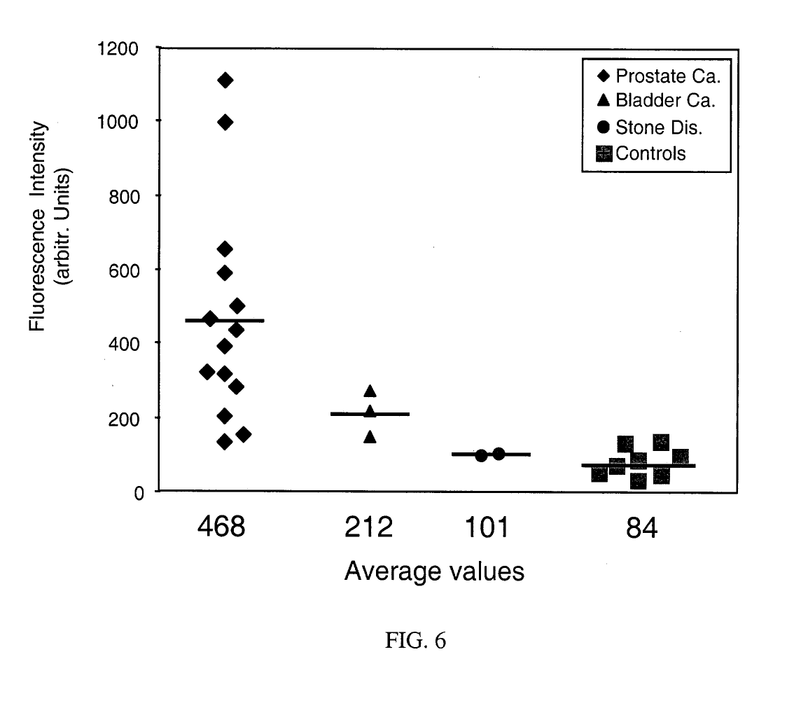

The results illustrated in FIG. 4A and FIG. 6 show that metalloproteinase “activity” is increased in urine of RCC patients. This does not necessarily mean that the “amount” of a metalloproteinase must be increased. It has been shown that the “amounts” of MP-2 and MP-9 are not consistently increased in urine of RCC patients compared to controls. MPs are generally synthesized and secreted by cells as inactive pro-enzymes. These inactive pro-enzymes would register positively in immunological assays (e.g. ELISA). MPs are normally activated by proteolytic cleavage of the pro-peptide but are then usually kept in an inactive state by binding to TIMPs. MPs that are bound to TIMPs would, however, also register positively in immunological assays. Even zymography can not distinguish between active and inactive MPs because during the course of the zymography experiment the MP-TIP interaction is broken resulting in artificial activation of normally inactive MPs. Furthermore, inactive pro-MPs are also activated during zymography because the pro-peptide is released from the active center of the enzyme during the required gel chromatography. Methods that detect the amount of metalloproteinases (such as immunological methods or zymography) do not measure the enzymatic activity of metalloproteinases in body fluids such as urine. While prior approaches to detect the amounts of MPs in urine have failed to be useful for diagnosing RCC, the methods of the present invention for detecting the proteolytic activity of metalloproteinases in urine have led to highly sensitive detection of RCC.

The diagnostic agent, method and kit of the present invention can be included as part of various immunoassay techniques, particularly ELISA and most preferably sandwich-type ELISA assays. Alternative immunoassay techniques such as immunoblotting, immunofluorescent, radio-immunoassay, fluorescence detection and/or enzyme assay methods are also contemplated.

The identification of metalloproteinases in clinical samples is preferably carried out using immunological techniques. The immunological techniques center around the use of specific antibody-antigen reactions which indicate a response to only specific antigens. Such MPs include, but are not limited to, gelatinase A (72 kDa type IV collagenase/gelatinase (MP2)), gelatinase B (92 kDa IV collagenase/gelatinase (MP-9), stromelysin (MP-3), and break down products of the proteinases and combinations thereof. For diagnostic purposes, it is of importance to note that the recognition of these antigenic substances is in both the free and complexed form.

In order to achieve this result, the invention includes a first substance having immunospecificity to free metalloproteinases and complexed metalloproteinases. In a preferred embodiment, monoclonal antibodies are prepared to have the desired immunospecificity. For example, monoclonal antibodies may be obtained from hybridomas obtained from mice immunized by injection of 72 kDa progelatinase/type IV procollagenase purified from human fibroblasts. See Birkedal-Hansen, et al. Biochemistry 27, 6751-6758 (1988). Many antibodies currently used can recognize free metalloproteinase. A non-limiting list of suitable antibodies include murine monoclonal anti-human 72 kDa or 92 kDa type IV collagenase/gelatinase antibodies, or murine monoclonal antibodies to stromelysins.

A preferred immunologic means of detecting metalloproteinases is the Enzyme-Linked Immunosorbent Assay (ELISA) method, and in particular the sandwich-type ELISA format. This assay method includes introducing a biological sample between a capture layer of antibodies and a detection layer of antibodies.

In this regard, diagnostic well plates, such as IMMULON II 96 well microtiter plates available from Dynatech, Alexandria, Va., are first coated with capture antibodies directed to MPs such as rabbit polyclonal antibodies to human 72 kDa gelatinase or 92 kDa progelatinase/type IV procollagenase. The capture antibodies are introduced into the wells in amounts ranging from about 10μ to about 200 μl with amounts of about 100 μl being preferred. The capture antibodies are preferably diluted in a suitable buffer such as 0.1 M NaHCO₃, pH 9.0 to about 1:200 concentration prior to introduction into the wells.

Binding of the capture antibodies to the well is carried out over a period of from about 4 to about 24 hours and preferably about 18 hours after inoculation at temperatures ranging from about 0° C. to about 10° C., and preferably about 4° C. Unbound antibodies are thereafter removed by vigorously inverting the plates. The wells containing the bound antibodies are next bathed with a bovine serum albumin/bicarbonate buffer to block excess binding sites on the wells. The bottom layer of the “sandwich” or capture layer is completed by washing the plate thoroughly with a buffer solution containing sodium phosphate, sodium chloride and Tween 20.

The ELISA technique further includes introducing a biological sample such as urine onto the capture antibody layer. The samples are preferably prepared by being diluted in an incubation-suitable buffer to about a 1:10 concentration. One such solution contains 50 mM sodium phosphate, 0.1 M sodium chloride, 0.02% Tween 20 and 0.1% bovine serum albumin (BSA). The samples are placed in the well, incubated at a temperature ranging from about 25° C. to about 37° C., and preferably at about 37° C. for a time period of from about 1 hour to about 4 hours and preferably about one hour.

The wells are incubated for about 1 hour and thereafter washed thoroughly and prepared using standard ELISA techniques known to the art, such as including the amplifying goat antibodies to mouse IgG and then alkaline phosphatase conjugated to streptavidin. The wells are then washed and 100 microliters of substrate p-nitrophenyl phosphate in buffer is added to generate a color reaction which is read at A₄₀₅. The results of the assay are obtained using any suitable reading device such as that available from BioTek of Winooski, Vt.

Other immunologic detection means can be readily adapted for use in connection with the diagnostic agent and method of the present invention. It is intended that all such alternative measuring and diagnostic means be included within the scope of the present invention.

With particular regard to the antibodies included herein, it will be appreciated by those of skill in the art that such antibodies, both monoclonal and polyclonal types are available from commercial sources such as Cell Tech Lmt. of Slough, England, or can be prepared using standard laboratory practices.

This invention is illustrated by examples set forth in the section that follows. This section is provided to aid in an understanding of the invention but is not intended to, and should not be construed to, limit in any way the invention as set forth in the claims which follow thereafter.

The term “subject,” as used herein, refers to a living animal or human in need of diagnosis or prognosis for, or susceptible to, a renal cell carcinoma. In preferred embodiments, the subject is a mammal, including humans and non-human mammals such as dogs, cats, pigs, cows, sheep, goats, horses, rats, and mice. In the most preferred embodiment, the subject is a human. The term “subject” does not preclude individuals that are entirely normal with respect to cancer (e.g. renal cell carcinoma) or normal in all respects. The subject may formerly have been treated surgically or by chemotherapy, and may be under treatment by hormone therapy or have been treated by hormone therapy in the past.

The term “patient,” as used herein, refers to a human subject who has presented at a clinical setting with a particular symptom or symptoms suggesting one or more diagnoses. A patient may be in need of further categorization by clinical procedures well-known to medical practitioners of the art (or may have no further disease indications and appear to be in any or all respects normal). A patient's diagnosis may alter during the course of disease progression, such as development of further disease symptoms, or remission of the disease, either spontaneously or during the course of a therapeutic regimen or treatment. In the invention here, a patient described in the Examples is listed with-other patients according to the most recent diagnosis of the medical condition, and any previous diagnoses, if different, are described in the text. Thus, the term “diagnosis” does not preclude different earlier or later diagnoses for any particular patient or subject. The term “prognosis” refers to assessment for a subject or patient of a probability of developing a condition associated with or otherwise indicated by presence of one or more enzymes in a biological sample, preferably in urine.

The term “electrophoresis” is used to indicate any separation system of molecules in an electric field, generally using an inert support system such as paper, starch gel, or preferably, polyacrylamide.

The term “zymography” is meant here to include any separations system utilizing a chemically inert separating or support matrix, that allows detection of an enzyme following electrophoresis, by exposing the matrix of the separations system to conditions that allow enzyme activity and subsequent detection. More narrowly, the term zymography designates incorporation of an appropriate substrate for the enzyme of interest into the inert matrix, such that exposing the matrix to the conditions of activity after the electrophoresis stop yields a system to visualize the precise location, and hence the mobility, of the active enzyme. By techniques well-known to the skilled artisan, the molecular weights of proteins are calculated based on mobilities derived from positions on a zymogram. Such techniques include comparison with molecular weight standards, the mobilities of which are determined from general protein stains or from pre-stains specific to those standards, and comparison with positive controls of purified isolated enzymes of interest, which are visualized by the technique of the zymogram, i.e., enzyme activity.

Zymography may be adapted to detection of a protease inhibitor in the biological sample. Since a variety of natural MP inhibitors are elaborated, such as TIMP-1 and TIMP-2, and are found to be deregulated during the development of renal cell carcinoma, the present invention includes detection of enzyme inhibitors as well as the enzymes of tissue remodelling. Thus for example, a “reporter enzyme” for which an enzyme's inhibitory activity is being measured, may be incubated with each biological sample obtained by subjects and patients, in one or more quantities corresponding to one or more aliquots of sample, prior to electrophoresis. This enzyme is omitted from one aliquot of the biological sample. The inhibitory presence in the sample is detected as disappearance or decrease of the reporter enzyme band from the developed zymogram. Alternatively, functional enzyme activity assays, which include in the reaction mix a known level of active enzyme, to which is added aliquots of experimental samples with putative inhibitory activity, can detect the presence of inhibitors. The zymogram as described in the Examples herein is developed by use of a general stain for protein, in this case, Coomassie Blue dye. The development is possible with general protein stains, e.g., Amido Black dye, and SYPRO Orange stain (Biorad Laboratories, Hercules, Calif. 94537). Further, enzyme activity may be detected by additional techniques beyond that of a clear zone of digestion in a stained matrix, for example, by absence of areas of radioactivity with a radiolabelled substrate, by change in mobility of a radio-labelled substrate, or by absence of or change in mobility of bands of fluorescence or color development with use of fluorogenic or chromogenic substrates, respectfully.

Quantitative densitometry can be performed with zymograms by placing the gel directly on an activated plate of a Molecular Dynamics phosphorimager (Molecular Dynamics, 928 East Arques Ave., Sunnyvale, Calif. 94086), or with a Datacopy G8 plate scanner attached to a MacIntosh computer equipped with an 8-bit videocard and McImage (Xerox Imaging Systems). Background measurements, areas of the gel separate from sample lanes, can similarly be scanned, and values subtracted from the readings for enzyme activities.

Another electrophoretically-based technique for analysis of a biological sample for presence of specific proteins is an affinity-based mobility alteration system (Lander, A., (1991), Proc. Natl. Acad. Sci. U.S., 88(7):2768-2772). An MP or other type of enzyme of interest might be detected, for example, by inclusion of a substrate analog that binds essentially irreversibly to the enzyme, hence decreasing the mobility. The affinity material is present during electrophoresis, and is incorporated into the matrix, so that detection of the enzyme of interest occurs as a result of alteration of mobility in contrast to mobility in the absence of the material. Yet another technique of electrophoretic protein separation is based on the innate charge of a protein as a function of the pH of the buffer, so that for any protein species, there exists a pH at which that protein will not migrate in an electric field, or the isoelectric point, designated pI Proteins of a biological sample, such as a urine sample, may be separated by isoelectric focussing, then developed by assaying for enzymatic activity for example by transfer to material with substrate, i.e., zymography. Electrophoresis is often used as the basis of immunological detections, in which the separation step is followed by physical or electrophoretic transfer of proteins to an inert support such as paper or nylon (known as a “blot”), and the blotted pattern of proteins may be detected by use of a specific primary binding (Western blot) by an antibody followed by development of bound antibodies by secondary antibodies bound to a detecting enzyme such as horseradish peroxidase. Additional immunological detection systems for TRAC enzymes are now described in detail below.

The term “antibody” as used herein is intended to include fragments thereof which are also specifically reactive with one of the components in the methods and kits of the invention. Antibodies can be fragmented using conventional techniques and the fragments screened for utility in the same manner as described above for whole antibodies. For example, F(ab)₂ fragments can be generated by treating an antibody with pepsin. The resulting F(ab)₂ fragment can be treated to reduce disulfide bridges to produce Fab fragments. The term “antibody” is further intended to include single chain, bispecific and chimeric molecules. The term “antibody” includes possible use both of monoclonal and polyclonal antibodies (Ab) directed against a target, according to the requirements of the application.

Polyclonal antibodies can be obtained by immunizing animals, for example rabbits or goats, with a purified form of the antigen of interest, or a fragment of the antigen containing at least one antigenic site. Conditions for obtaining optimal immunization of the animal, such as use of a particular immunization schedule, and using adjuvants e.g. Freund's adjuvant, or immunogenic substituents covalently attached to the antigen, e.g. keyhole limpet hemocyanin, to enhance the yield of antibody titers in serum, are well-known to those in the art. Monoclonal antibodies are prepared by procedures well-known to the skilled artisan, involving obtaining clones of antibody-producing lymphocyte, i.e. cell lines derived from single cell line isolates, from an animal, e.g. a mouse, immunized with an antigen or antigen fragment containing a minimal number of antigenic determinants, and fusing said clone with a myeloma cell line to produce an immortalized high-yielding cell line. Many monoclonal and polyclonal antibody preparations are commercially available, and commercial service companies that offer expertise in purifying antigens, immunizing animals, maintaining and bleeding the animals, purifying sera and IgG fractions, or for selecting and fusing monoclonal antibody producing cell lines, are available.

Specific high affinity binding proteins, that can be used in place of antibodies, can be made according to methods known to those in the art. For example, proteins that bind specific DNA sequences may be engineered (Ladner, R. C., et. al., U.S. Pat. No. 5,096,815), and proteins that bind a variety of other targets, especially protein targets (Ladner, R. C., et. al., U.S. Pat. No. 5,233,409; Ladner, R. C., et. al, U.S. Pat. No. 5,403,484) may be engineered and used in the present invention for covalent linkage to a chelator molecule, so that a complex with a radionucleotide may be formed under mild conditions. Antibodies and binding proteins can be incorporated into large scale diagnostic or assay protocols that require immobilizing the compositions of the present invention onto surfaces, for example in multi-well plate assays, or on beads for column purifications.

General techniques to be used in performing various immunoassays are known to those of ordinary skill in the art. Moreover, a general description of these procedures is provided in U.S. Pat. No. 5,051,361 which is incorporated herein by reference, and by procedures known to the skilled artisan, and described in manuals of the art (Ishikawa, E., et. al. (1988), Enzyme Immunoassay Igaku-shoin, Tokyo, NY; Hallow, E. and D. Lane, Antibodies: A Laboratory Manual CSH Press, NY). Examples if several immunoassays are discussed here.

Radioimmunoassays (RIA) utilizing radioactively labeled ligands, are available to measure presence of MP's as antigenic material. A fixed quantity of labeled MP antigen competes with unlabeled antigen from the sample for a limited number of antibody binding sites. After the bound complex of labeled antigen-antibody is separated from the unbound (free) antigen, the radioactivity in the bound fraction, or free fraction, or both, is determined in an appropriate radiation counter. The concentration of bound labeled antigen is inversely proportional to the concentration of unlabeled antigen present in the sample. The antibody to MP can be in solution, and separation of free and bound antigen MP can be accomplished using agents such as charcoal, or a second antibody specific for the animal species whose immunoglobulin contains the antibody to MP. Alternatively, antibody to MP can be attached to the surface of an insoluble material, which in this case, separation of bound and free MP is performed by appropriate washing.

Immunoradiometric assays (IRMA) are immunoassays in which the antibody reagent is radioactively labeled. An IRMA requires the production of a multivalent MP conjugate, by techniques such as conjugation to a protein e.g., rabbit serum albumin (RSA). The multivalent MP conjugate must have at least 2 MP residues per molecule and the MP residues must be of sufficient distance apart to allow binding by at least two antibodies to the MP. For example, in an IRMA the multivalent MP conjugate can be attached to a solid surface such as a plastic sphere. Unlabeled “sample” MP and antibody to MP which is radioactively labeled are added to a test tube containing the multivalent MP conjugate coated sphere. The MP in the sample competes with the multivalent MP conjugate for MP antibody binding sites. After an appropriate incubation period, the unbound reactants are removed by washing and the amount of radioactivity on the solid phase is determined. The amount of bound radioactive antibody is inversely proportional to the concentration of MP in the sample.

Other preferred immunoassay techniques use enzyme labels such as horseradish peroxidase, alkaline phosphatase, luciferase, urease, and β-galactosidase. For example, MP's conjugated to horseradish peroxidase compete with free sample MP's for a limited number of antibody combining sites present on antibodies to MP attached to a solid surface such as a microtiter plate. The MP antibodies may be attached to the microtiter plate directly, or indirectly, by first coating the microtiter plate with multivalent MP conjugates (coating antigens) prepared for example by conjugating M with serum proteins such as rabbit serum albumin (RSA). After separation of the bound labeled MP from the unbound labeled MP, the enzyme activity in the bound fraction is determined calorimetrically, for example by a multi-well microtiter plate reader, at a fixed period of time after the addition of horseradish peroxidase chromogenic substrate.

Alternatively, the antibody, attached to a surface such as a microtiter plate or polystyrene bead, is incubated with an aliquot of the biological sample. MP present in the fluid will be bound by the antibody in a manner dependent upon the concentration of MP and the association constant between the two. After washing, the antibody/MP complex is incubated with a second antibody specific for a different epitope on MP distal enough from the MP-specific antibody binding site such that steric hindrance in binding of two antibodies simultaneously to MP may be accomplished. For example, the second antibody may be specific for a portion of the proenzyme sequence. The second antibody can be labeled in a manner suitable for detection, such as by radioisotope, a fluorescent compound or a covalently linked enzyme. The amount of labeled secondary antibody bound after washing away unbound secondary antibody is proportional to the amount of MP present in the biological sample.

The above examples of preferred immunoassays describe the use of radioactively and enzymatically labeled tracers. Assays also may include use of fluorescent materials such as fluorescein and analogs thereof, 5-dimethylaminonaphthalene-1-sulfonyl derivatives, rhodamine and analogs thereof, coumarin analogs, and phycobiliproteins such as allophycocyanin and R-phycoerythrin; phosphorescent materials such as erythrosin and europium; luminescent materials such as luminol and luciferin; and sols such as gold and organic dyes. In one embodiment of the present invention, the biological sample is treated to remove low molecular weight contaminants.

In one embodiment of the present invention, the biological sample is treated to remove low molecular weight contaminants, for example, by dialysis. By the term “dialysis” this invention includes any technique of separating the enzymes in the sample from low molecular weight contaminants. The Examples use Spectra/Por membrane dialysis tubing with a molecular weight cut-off (MWCO) of 3,500, however other products with different MWCO levels are functionally equivalent. Other products include hollow fiber concentration systems consisting of regenerated cellulose fibers (with MWCO of 6,000 or 9,000) for larger volumes; a multiple dialyzer apparatus with a sample size for one to 5 ml; and multiple microdialyzer apparatus, convenient for samples in plates with 96 wells and MWCOs at 5,000, 8,000 and 10,000, for example. These apparatuses are available from PGC Scientific, Gaithersburg, Md., 20898. Those with skill in the art will appreciate the utility of multiple dialysis units, and especially suitable for kits for reference lab and clinic usage. Other equivalent techniques include passage through a column holding a resin or mixture of resins suitable to removal of low molecular weight materials. Resins such as BioGel (BioRad, Hercules, Calif.) and Sepharose (Pharmacia, Piscataway, N.J.) and others are well-known to the skilled artisan. The technique of dialysis, or equivalent techniques with the same function, are intended to remove low molecular weight contaminants from the biological fluids. While not an essential component of the present invention, the step of removal of such contaminants facilitates detection of the disorder associated enzymes in the biological samples.

The invention is further illustrated by the following examples, which should not be construed as further limiting. The contents of all references, pending patent applications and published patents, cited throughout this application are hereby expressly incorporated by reference.

An important aspect of the present invention is that urine from patients with renal cell carcinoma appears to contain elevated levels of MP activity, which results in the degradation of normally excreted, full-length ECM proteins. Accordingly, the absence of ECM proteins and presence of MP activity can be exploited to develop a noninvasive screening assay to indicate the presence of renal cell carcinoma.

A large number of MPs are known to proteolyse specifically various extracellular matrix extracellular matrix proteins under normal and pathological conditions. MPs have an important role in tumor invasion and metastatsis. There is evidence for increase MP expression in renal cell carcinoma. MP-9 was reported to be significantly elevated in tumor tissue of patients with renal cell carcinoma, whereas MP-2 was reportedly unaffected. In contrast, it has been found that MP-2 and MP-9 are elevated in renal cell carcinoma tumor tissue and their expression level correlates with tumor aggressiveness. Urine was not examined in either study, nor was the expression of MPs other than MP-2 and MP-9.

TABLE 1
Semiquantification of immunoblot analysis of urinary full-length ECM proteins

| | Control* | | | | Renal Cell Ca* | | | | | |
|---|---|---|---|---|---|---|---|---|---|---|
| | | | | | Pathological | Fuhrman | | | | Collagen |
| Sex | Laminin | Fibronectin | Collagen IV | Sex | Tumor Type | Grade | TNM Stage | Laminin | Fibronectin | IV |
| M | +++ | ++ | +++ | M | Clear cell + | 3 | Tb3N2M1 | − | − | − |
| | | | | | granula | | | | | |
| M | +++ | ++++ | +++ | M | Clear cell + | 3 | Tb3N2M1 | − | − | − |
| M | ++ | +++ | ++++ | M | Clear cell + | ¾ | T1N0M0 | − | − | − |
| | | | | | papillary | | | | | |
| M | +++ | +++ | +++++ | M | Papillary | 2 | T2N0M0 | + | − | − |
| M | +++ | +++ | +++ | M | Clear cell | 2 | T2N0M0 | − | + | + |
| M | ++++ | +++ | ++++ | M | Papillary | 3 | T1N0M0 | − | − | − |
| M | +++ | ++ | ++ | M | Clear cell + | 2 | T2N0M0 | − | − | − |
| | | | | | papillary | | | | | |
| M | ++ | ++ | ++ | M | Clear cell | 2 | T2N0M0 | − | − | + |
| M | +++ | ++++ | ++++ | M | Clear cell | 2 | T1N0M0 | − | − | − |
| M | ++ | + | ++ | M | Clear cell | 2 | T1N0M0 | − | − | − |
| M | + | + | + | M | Clear cell | 3 | T3N0M0 | − | + | − |
| M | +++ | +++ | +++ | M | Papillary | 2 | T1N0M0 | − | − | − |
| M | +++ | ++++ | +++ | M | Clear cell | 2 | T3aN0M0 | − | − | − |
| M | ++ | ++ | +++ | F | Clear cell | 2 | T1N0M0 | − | − | − |
| F | ++ | +++ | +++ | F | Clear cell | 3 | T1N0M0 | − | − | − |
| F | ++ | +++ | +++ | F | Clear cell | 3 | T3bN2M1 | − | − | − |
| F | ++ | + | − | F | Clear cell | 3 | T3bN2M1 | − | − | − |
| F | + | + | + | F | Clear cell | 2 | T3BN2M1 | − | − | − |
| F | − | − | + | F | Clear cell | ½ | T1N0M0 | − | − | − |
| F | − | ++ | + | F | Clear cell | 2 | T1N0M0 | − | − | − |
| F | ++ | +++ | ++++ | F | Clear cell + | 4 | T1N0M0 | − | − | − |
| | | | | | sarcomatoid | | | | | |
| F | +++ | ++++ | +++ | F | Clear cell | 2 | T1N0M0 | − | − | − |

*Since signal intensities in patients and controls far exceeded the linear range of film detection, signals were analyzed semiquantitatively by comparing multiple exposures and defining a range from undetectable (−) to maximum signal (+++++).

Patients with proteinuria or hematuria were excluded from analysis in order to render it unlikely that the increased level of MP activity in urine was due to leakage from serum caused by compromised renal barrier function. It is possible that affected MPs reach the urine by glomerular filtration or direct secretion from the tumor into the urinary space. The latter possibility may be expected to lead to much more efficient MP excretion.

Experimental results indicate that analysis of urinary ECM proteins allows the detection of renal cell carcinoma with high sensitivity. Table 1 illustrate the semiquantitative analysis of these results with pathological findings.

Although as shown in FIG. 4, MP activity measured by the fluorescence based assay was an average of 3.4-fold increased in the renal cell carcinoma population compared with controls, there was a greater overlap in the 2 groups compared with the analysis of ECM proteins. Higher sensitivity of the ECM assay may have been due to the fact that the urinary ECM proteins are subjected to proteolytic activity at body temperature in the urinary tract for an extended period before voiding. This scenario would cause “in-body enzymatic signal amplification” and, therefore, higher sensitivity. However, the simplicity of the fluorescence based activity assay makes it much more suitable for rapid, high throughput analyses and optimization of this assay may result in improved differentiation of patients from controls.

Even at early stages of disease patients with renal cell carcinoma excrete significantly elevated levels of MP activity with the urine, causing the degradation of full-length ECM proteins. The analysis of ECM proteins and/or MP activity in urine has high potential as a marker to allow noninvasive, high throughput screening for the early detection of renal cell carcinoma.

A total of 36 patients with renal cell carcinoma who presented for surgery at the urological institute at the Cleveland Clinic institution between July 2001 and January 2002 were entrolled in this study. Patients with other kidney related diseases or who received medications that may interfere with normal kidney functions were excluded from analysis. Also excluded from the study were patients with proteinuria or hematuria because this condition would obscure the origin of any potential renal cell carcinoma specific marker. Proteinuria and hematuria were assessed using Labstix Reagent Strips (Bayer Corp., Elkhart, Ind.). Patient age was 25 to 78 years (mean age 54.1). Spot urine samples (30 to 100 ml) were collected once 2 to 4 weeks before surgery. The diagnosis was confirmed by pathological findings. All patients provided written consent. Control urine samples were also spot voids obtained anonymously from 39 apparently healthy volunteers 23 to 63 years old (mean age 44.5).

Urine samples were collected in sterile containers and processed immediately. The study populations were divided into 2 groups depending on the intended experimental use. Urine from these groups was concentrated by 2 methods. In group 1 it was dialyzed against 50 mM. ammonium bicarbonate using 12 to 14 kDa cutoff dialysis tubing (Invitrogen, Carlsbad, Calif.) after adding a protease inhibitor cocktail, 10 mM ethylenediamine tetraacetic acid and 50 mM. ammomium bicarbonate. Samples were tetraacetic acid and 50 mM ammonium bicarbonate. Samples were lyophilized and reconstituted in water, resulting in a 30-fold concentration, and then aliquoted and stored at −80° C. for experiments. Urine from group 2 was immediately concentrated 20-fold by ultrafiltration using Centriprep (Millipore, Bedford, Mass.) centrifugal filter devices with a 12 to 14 kDa. cutoff and stored at −80° C. for activity based assays. All subsequent assays were standardized by an equal volume of the original urine.

Urine samples (20 μl) concentrated 30-fold from patients and controls were separated on 6% polyacrylamide gels. Proteins were electro-transferred into nitrocellulose membranes and probed with primary antibodies for laminin, fibronectin (Sigma Chemical Co., St. Louis, Mo.) or collagen IV (Rockland Immunochemicals, Gilbertsville, Pa.) and subsequently with secondary antibodies coupled to horseradish peroxidase. Bands were visualized by enhanced chemiluminescense. For semiquantitative analysis Western blots were encoded and 2 researchers not associated with this study were asked to assign to each signal a value of between − and +++++. Discrepancies were resolved by discussion.

Gelatin (1 mg/ml.) was incorporated into 10% sodium dodecyl sulfate-polyacrylamide gel ectrophoresis (SDS-PAGE) gels. Urine samples (15 μl.) concentrated 20-fold were mixed with SDS-PAGE sample buffer without dithiothreitol and loaded directly onto gels. After electrophoresis gels were incubated for renaturation at room temperature in a buffer containing 50 mM. tris, pH 7.5, 150 mM NaCl, 5 mM CaCl₂ and 2.5% Triton-X100 overnight. Gels were further incubated in the same buffer lacking Triton-X100 for 16 to 18 hours. Afterward gels were stained with Coomassie blue to visualize white negative bands indicating the presence of protease activity.

For each experiment 0.3 μg. purified human fibronectin (Roche Molecular Biochemical, Indianapolis, Ind.) were incubated with 150 μl. 20-fold concentrated renal cell carcinoma or control urine at 37° C. At different time points (0, 1, 3, 6 0r 16 hours) 25 μl. were removed from the reaction and analyzed from the disappearance of full-length fibronectin by SDS-PAGE and Western blot, as described.

) Urine samples (50 μl.) concentrated 20-fold were mixed in duplicate with 2 μg fluorescein conjugated collagen IV substrate (Molecular Probes, Eugene, Oreg.) and 10× reaction buffer containing 0.5 M. tris-HCl, 1.5 M NaCl, 50 mM CaCl₂ and 2 mM. sodium azide, pH 7.6, into 96-well plates in a total volume of 200 μl. Plates were incubated at room temperature for 20 hours. Proteolytic activity was monitored using a fluorescence microplate reader and background was subtracted. As a control, metalloproteinase activity was inhibited by including 2.5 mM 1,10-phenathroline. Standard curves were measured using collagenase-IV from Clostridium histolyticum (Molecular Probes).

Normal renal epithelial cells have a polarized morphology and secrete most proteins in strictly polarized fashion apically into the tubule lumen or basolaterally into the interstitial space. In contrast, carcinoma cells (epithelial derived malignant tumors, including renal cell carcinoma) have partially or completely lost cell polarity to a degree that usually correlates with the degree of malignancy.

ECM proteins laminin, collagen IV and fibronectin, which are known to be secreted basolaterally by renal epithelial cells. Urine samples from 22 preoperative patients with renal cell carcinoma and 22 healthy controls were analyzed. Patients with renal cell carcinoma who had hematuria or proteinuria were excluded from analysis. These 3 ECM proteins were significantly decreased or absent in renal cell carcinoma urine samples compared with controls. FIG. 1A shows examples of Western blot analyses indicating that the full-length forms of all 3 ECM proteins were dramatically reduced in renal cell carcinoma urine.

Table 1 lists semiquantitative analysis of these results with pathological findings. Defining the absence (−) of at lease 2 ECM proteins as indicative of renal cell carcinoma, this analysis would detect renal cell carcinoma (95%). Only 1 of 22 controls (4.5%) showed 2 undetectable ECM proteins. The absence of ECM proteins was detected even in 11 patients with nonmetastatic disease of the lowest clinical stage (T1N0M0) which was typically asymptomatic and discovered incidentally. This finding indicates that the analysis of urinary ECM proteins may have value for the early detection of renal cell carcinoma.

For this analysis all specimens were collected at random times and standardization was done by using an equal volume of urine. To exclude the possibility that the excretion of ECM proteins may vary depending on the time of specimen collection successive samples from healthy volunteers standaradized by equal volume were analyzed for full-length collagen IV (FIG. 1B). Collagen IV excretion varied only modestly between sample collection times. Therefore, the time of specimen collection is unlikely to account for the observed differences in patients with renal cell carcinoma and controls.

FIG. 2 shows electrophoretic patterns of urinary proteins of 12 patients with renal cell carcinoma and 12 controls. While changes in the band patters in some patients and controls were detectable, differences were not observed consistently enough to be a reliable marker. No general protein degradation was apparent in renal cell carcinoma urine. This result indicates that urinary EMC proteins are specifically affected in renal cell carcinoma.

The absence of ECM proteins was due to increased excretion of MPs in the urine of patients with renal cell carcinoma. Incubated purified human fibronectin with equivalent amounts of urine from controls or patients were prepared. FIG. 3 shows that patient but not control urine contained proteolytic activity that led to significant degradation of fibronectin. Proteolytic activity could be inhibited by the zinc chelator, 1,10-phenanthroline, indicating that the involved enzymes belonged to the metal-loproetinase family (FIG. 4.). To investigate further the excretion of MPs urine specimens were analyzed by gelatin zymography. FIG. 5 shows that renal cell carcinoma urine contained elevated levels of gelatinases compared with controls.

An example of a screening assay for urinary MP activity that may be suitable for screening large subject populations was demonstrated. Urine samples of 18 patients with renal cell carcinoma and 17 controls (different groups from the aforementioned analyses) were incubated with collagen IV heavily labeled with fluorescein, so that fluorescence was quenched. Proteolytic digestion results in fluorescence de-quenching due to the release of highly fluorescent peptides. The increase in fluorescence is proportional to the proteolytic activity and it is measured in a microtiter plate fluorscense reader. FIG. 4 shows that the average activity measured by this assay was significantly increased by approximately 4-fold in the renal cell carcinoma population (t test p<0.00001).

FIG. 6 illustrates detection of MP activity using a fluorescence based microtiter plate assay. Concentrated urine samples from patients and controls different from those shown in Table 1 and FIG. 4A were mixed in 96-well plates in duplicate with reaction buffer containing fluorescent substrate, namely collagen IV modified with fluorescein. Proteolytic activity was indicated by increased fluorescence, which was monitored with a fluorescence microplate reader. Background was subtracted and average fluorescence values were plotted for patients with different urological cancers, a urological disease, and healthy controls. The amount of active metalloproteinases detected by these methods may be compared to the mean amount of active metalloproteinases in a control group of healthy patients or to the normal population as shown in FIG. 6. An increase in the amount of active metalloproteinases in the subject's urine being an indication of an increased probability of a urological cancer. The results show that the assay may be used to detect the absence or decrease of active metalloproteinases in biological fluids, particularly in urine in order to facilitate the diagnosis of the subject for cancer, particularly urological cancers such as prostate, kidney, and bladder cancer. The presence or absence of extracellular matrix proteins or their proteolytic degradation products in the urine of the subject may also be used to facilitate the diagnosis of cancer in the subject.

While this invention has been described with an emphasis upon preferred embodiments, it will be obvious to those of ordinary skill in the art that variations of the preferred compounds and methods may be used and that it is intended that the invention may he practiced otherwise than as specifically described herein.

All of the references cited herein and appended hereto, including patents, patent applications, literature publications, and the like, are hereby incorporated in their entireties by reference.

What is claimed:

1. A method of diagnosing cancer in a urine sample from a subject, comprising:
detecting a presence of an active metalloproteinase in the urine sample; and
correlating the presence of the active metalloproteinase to a presence of cancer in the subject.

2. The method of claim 1 wherein the step of detecting the presence of the active metalloproteinase comprises detecting the presence or absence of an extracellular matrix protein in said urine sample.

3. The method of claim 2 wherein the extracellular proteins include one extracellular matrix protein chosen from the group consisting of laminin, collagen IV, or fibronectin.

4. The method of claim 2 wherein at least two extracellular matrix proteins being absent facilitating the diagnosis of renal cell carcinoma in a subject.

5. The method of claim 1 wherein the step of detecting the presence of the active metalloproteinase comprises detecting the presence or absence of a degradation product of an extracellular matrix protein in said urine sample.

6. The method of claim 5 further comprising the act of incubating the urine sample from the subject with an added extracellular matrix protein following the act of obtaining the urine sample from the subject.

7. The method of claim 1 wherein the step of detecting the presence of the active metalloproteinase comprises detecting the amount of metalloproteinase in said sample.

8. The method of claim 1 wherein the step of detecting the presence of the active metalloproteinase comprises detecting the presence or absence of an active metalloproteinase in said sample.

9. The method as in any of claims, 1 wherein the urine is preconcentrated.

10. The method of claim 1, wherein the metalloproteinase is in its proenzyme form.

11. The method as in any of claims 1 wherein the subject has previously been treated surgically to treat a urological cancer.

12. The method of claim 1, wherein the extracellular matrix proteins are detected by an electrophoretic pattern and immunological detection.

13. The method of claim 1, wherein the extracellular matrix proteins are detected using antibodies specific for a proteolytic site on said proteins.

14. The method of claim 1, wherein the metalloproteinase is detected by an electrophoretic pattern.

15. The method of claim 1, wherein the metalloproteinase is detected immunochemically.

16. The method of claim 1, wherein the metalloproteinase is detected by a radio-immune assay.

17. The method of claim 1, wherein the metalloproteinase is detected by an enzyme-linked immunosorbant assay.

18. The method of claim 1 wherein, the metalloproteinase is detected by a fluorescence-based microtiter plate assay.

19. The method of claim 18 wherein the assay comprises fluorescein conjugated college IV.

20. A method of detecting a urological cancer in a subject comprising:
determining an amount of active metalloproteinase in a urine sample from said subject; and
comparing said determined amount active metalloproteinases to the mean amount the metalloproteinases in urine of a normal population, an increase in the amount of active metalloproteinases in the subject's urine being an indication of increased probability of a urological cancer.

21. A method of detecting a urological cancer in a subject comprising:
determining an amount of active metalloproteinase in a urine sample from a subject; and
comparing said determined amount of active metalloproteinase to the mean amount of the metalloproteinase in urine of a normal population, an increase in the amount of active metalloproteinase in the subject's urine being an indication of an increased probability of a urological cancer.

22. A method for monitoring the state of a urological cancer in a subject, comprising:
establishing a baseline amount of extra-cellular matrix protein in urine from a subject;
measuring the amount of extra-cellular matrix protein in a sample of urine from the subject; and
comparing the measured amount of extra-cellular matrix protein in the sample of urine to the baseline amount of extra-cellular matrix protein from the subject, whereby a reduced level of extracellular matrix protein in the urine sample compared to the baseline amount of extracellular matrix protein indicates a deteriorating condition while an increased level indicates an improving state of the urological cancer in the subject.

23. The method of claim 21 wherein a change in the amount of at least two of the extracellular matrix proteins in the urine of the subject being an indication of a change in the state of the urological cancer in the subject.

24. The method of claim 22 wherein the extracellular protein includes collegen-IV.

25. The method of claim 22 wherein the extracellular protein includes laminin.

26. The method of claim 22 wherein the extracellular protein includes fibronectin.

27. A kit for detecting a urological cancer in a subject comprising:
a reagent including a buffer and a fluorescent substrate;
well plates; and
a sterile container for combining the reagent with urine from the subject to form a mixture, said mixture deposited into the well plates, wherein the presence of fluorescence of the mixture in the well plate used as an indication that the subject has a urological cancer.

28. The kit of claim 27 wherein said fluorescent substrate is fluorescein conjugated collagen IV.

29. A kit for detecting a urological cancer in a subject comprising:
a reagent including antibodies against an extracellular matrix protein; and
a sterile container for combining the reagent with urine from the subject to form a mixture, said mixture deposited onto a separating media to identify said proteins for use as an indication that the subject has a urological cancer.

30. The kit of claim 29 wherein said proteins are identified by immunoblotting.
